(19) United States
(12) Patent Application Publication
(10) Pub. No.: US 2012/0310087 A1
(43) Pub. Date: Dec. 6, 2012

(54) ULTRASONIC DIAGNOSIS APPARATUS, OPERATION METHOD OF THE SAME, AND COMPUTER READABLE RECORDING MEDIUM

(75) Inventors: Hironaka MIYAKI, Tokyo (JP); Tadaaki KAMBARA, Tokyo (JP); Yasuhiro WADA, Tokyo (JP)

(73) Assignee: OLYMPUS MEDICAL SYSTEMS CORP., Tokyo (JP)

(21) Appl. No.: 13/469,191

(22) Filed: May 11, 2012

Related U.S. Application Data
(63) Continuation of application No. PCT/JP2011/076603, filed on Nov. 11, 2011.

(30) Foreign Application Priority Data
Nov. 11, 2010 (JP) 2010-253289

(51) Int. Cl. A61B 8/08; A61B 8/14

(52) U.S. Cl. 600/440; 600/442

(57) ABSTRACT

An ultrasonic diagnosis apparatus includes a frequency analyzing unit that analyzes frequencies of received ultrasonic sound waves and calculates a frequency spectrum; a feature data extracting unit that performs approximation and correction of the frequency spectrum so that there is a decrease in the contribution of attenuation, and extracts feature data of the specimen; a storing unit used to store feature data of frequency spectrums, each being extracted based on ultrasonic sound waves reflected from one of a plurality of known specimens, and tissue characterizations of the known specimens in a corresponding manner; and a tissue characterization determining unit that determines tissue characterization of a predetermined area of the specimen by referring to feature data, which is stored by the memory unit in a corresponding manner to the tissue characterizations of the plurality of known specimens, and by referring to the feature data extracted by the feature data extracting unit.

### CROSS REFERENCES TO RELATED APPLICATIONS

This application is a continuation of PCT international application Ser. No. PCT/JP2011/076603 filed on Nov. 11, 2011 which designates the United States, incorporated herein by reference, and which claims the benefit of priority from Japanese Patent Applications No. 2010-253289, filed on Nov. 11, 2010, incorporated herein by reference.

### BACKGROUND OF THE INVENTION

1. Field of the Invention

The present invention relates to an ultrasonic diagnosis apparatus, an operation method of the ultrasonic diagnosis apparatus, and a computer readable recording medium for enabling determination of tissue characterizations of specimens using ultrasonic sound waves.

2. Description of the Related Art

Typically, in order to perform screening for breast cancer using ultrasonic sound waves, a technology called ultrasonic elastography is known (for example, see WO/2005/122906). The ultrasonic elastography is a technology which makes use of the fact that cancer tissues or tumor tissues inside a body have different hardness depending on the disease progression or depending on the body nature. In this technology, while continually applying external compression to the screening location, the strain amount or the degree of elasticity of the body tissues at the screening location is measured using ultrasonic sound waves, and the measurement result is displayed in the form of cross-sectional images.

### SUMMARY OF THE INVENTION

An ultrasonic diagnosis apparatus according to the present invention transmits ultrasonic sound waves to a specimen and receives ultrasonic sound waves reflected from the specimen, and that determines tissue characterization of the specimen based on the received ultrasonic sound waves, the ultrasonic diagnosis apparatus including: a frequency analyzing unit that analyzes frequencies of the received ultrasonic sound waves and calculates a frequency spectrum; a feature data extracting unit that performs attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracts feature data of the specimen; a storing unit that is used to store feature data of frequency spectrums, each being extracted based on ultrasonic sound waves reflected from one of a plurality of known specimens, and tissue characterizations of the known specimens in a corresponding manner; and a tissue characterization determining unit that determines tissue characterization of a predetermined area of the specimen by referring to feature data, which is stored by the memory unit in a corresponding manner to the tissue characterizations of the plurality of known specimens, and by referring to the feature data extracted by the feature data extracting unit.

An operation method of an ultrasonic diagnosis apparatus according to the present invention, which transmits ultrasonic sound waves to a specimen and receives ultrasonic sound waves reflected from the specimen, and which determines tissue characterization of the specimen based on the received ultrasonic sound waves, includes: calculating that includes analyzing frequencies of the received ultrasonic sound waves and calculating a frequency spectrum by a frequency analyzing unit; extracting that includes performing attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracting feature data of the specimen by a feature data extracting unit; and determining that includes determining tissue characterization of a predetermined area of the specimen by referring to feature data of frequency spectrums, each being extracted based on ultrasonic sound waves reflected from one of a plurality of known specimens, and by referring to the feature data extracted by the feature data extracting unit by a tissue characterization determining unit.

A non-transitory computer readable recording medium according to the present invention has an executable program stored thereon, wherein the program instructs a processor to perform: calculating that includes analyzing frequencies of the received ultrasonic sound waves and calculating a frequency spectrum by a frequency analyzing unit; extracting that includes performing attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracting feature data of the specimen by a feature data extracting unit; and determining that includes determining tissue characterization of a predetermined area of the specimen by referring to feature data of frequency spectrums, each being extracted based on ultrasonic sound waves reflected from one of a plurality of known specimens, and by referring to the feature data extracted by the feature data extracting unit by a tissue characterization determining unit.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram illustrating a configuration of an ultrasonic diagnosis apparatus according to a first embodiment of the present invention;

FIG. 2 is a diagram illustrating a relationship between gains and reception depths of echo signals for B-mode images;

FIG. 3 is a diagram illustrating a relationship between gains and reception depths of echo signals for processing;

FIG. 4 is a flowchart explaining an overview of the operations performed by the ultrasonic diagnosis apparatus according to the first embodiment of the present invention;

FIG. 5 is a diagram illustrating an example of a B-mode image displayed by a display unit of the ultrasonic diagnosis apparatus according to the first embodiment of the present invention;

FIG. 6 is a flowchart explaining an overview of the operations performed by a frequency analyzing unit of the ultrasonic diagnosis apparatus according to the first embodiment of the present invention;

FIG. 7 is a diagram that schematically illustrates data arrangement of a single acoustic ray;

FIG. 8 is a diagram illustrating an example (first example) of the frequency spectrum calculated by the frequency analyzing unit of the ultrasonic diagnosis apparatus according to the first embodiment of the present invention;

FIG. 9 is a diagram illustrating an example (second example) of the frequency spectrum calculated by the frequency analyzing unit of the ultrasonic diagnosis apparatus according to the first embodiment of the present invention;

FIG. 10 is a diagram illustrating a new straight line that is determined from the feature data obtained upon performing attenuation correction of the feature data related to a straight line illustrated in FIG. 7;

FIG. 11 is a flowchart explaining an overview of the operations performed by a tissue characterization determining unit of the ultrasonic diagnosis apparatus according to the first embodiment of the present invention;

FIG. 12 is a diagram illustrating an example of the feature data space set by the tissue characterization determining unit of the ultrasonic diagnosis apparatus according to the first embodiment of the present invention;

FIG. 13 is a diagram illustrating a display example of the determination result display image displayed by the display unit of the ultrasonic diagnosis apparatus according to the first embodiment of the present invention;

FIG. 14 is a diagram explaining the effect of attenuation correction performed by the ultrasonic diagnosis apparatus according to the first embodiment;

FIG. 15 is a diagram illustrating a display example (first example) of a tissue characterization weighted image in which a color image is used;

FIG. 16 is a diagram that schematically illustrates a black-and-white image of the image illustrated in FIG. 15;

FIG. 17 is a diagram illustrating a display example (second example) of a tissue characterization weighted image in which a color image is used;

FIG. 18 is a diagram that schematically illustrates a black-and-white image of the image illustrated in FIG. 17;

FIG. 19 is a diagram illustrating a display example (third example) of a tissue characterization weighted image in which a color image is used;

FIG. 20 is a diagram that schematically illustrates a black-and-white image of the image illustrated in FIG. 19;

FIG. 21 is a flowchart explaining an overview of the operations performed by the ultrasonic diagnosis apparatus according to a second embodiment of the present invention;

FIG. 22 is a diagram that schematically illustrates an overview of the attenuation correction performed by the ultrasonic diagnosis apparatus according to the second embodiment; and

FIG. 23 is a diagram explaining the overview of the tissue characterization determining operation performed by the tissue characterization determining unit of the ultrasonic diagnosis apparatus according to a fifth embodiment of the present invention.

### DETAILED DESCRIPTION OF THE PREFERRED EMBODIMENTS

Exemplary illustrative embodiments of the present invention (hereinafter, referred to as “embodiments”) are explained below in detail with reference to the accompanying drawings.

### First Embodiment

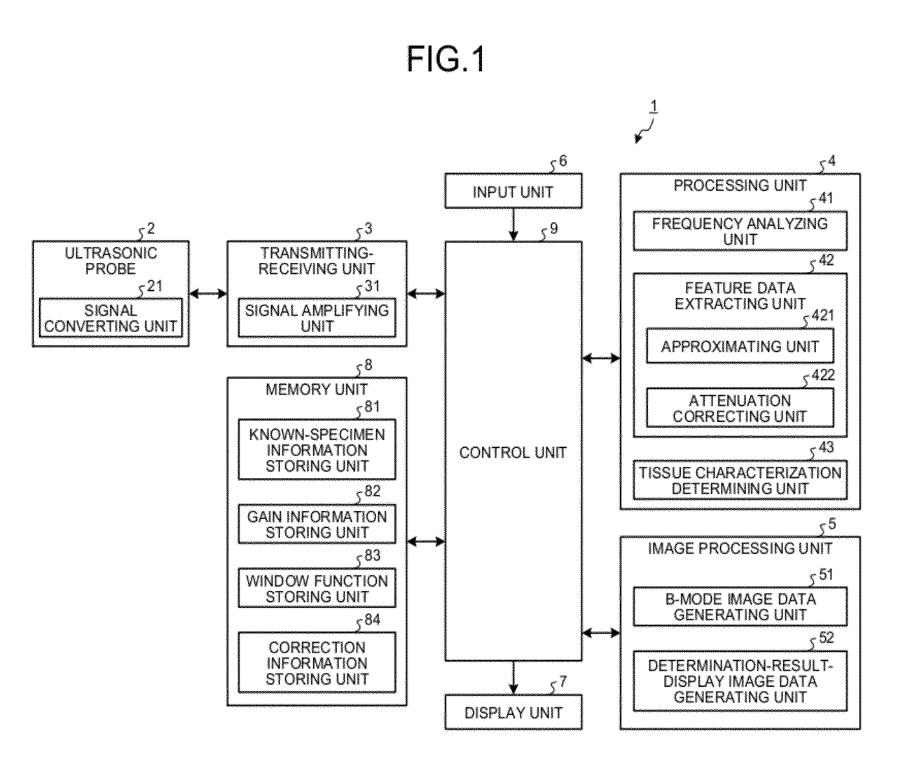

FIG. 1 is a block diagram illustrating a configuration of an ultrasonic diagnosis apparatus according to a first embodiment of the present invention. An ultrasonic diagnosis apparatus 1 illustrated in FIG. 1 is an apparatus for determining tissue characterizations of target specimens for diagnosis using ultrasonic sound waves.

The ultrasonic diagnosis apparatus 1 includes an ultrasonic probe 2 that outputs an ultrasonic pulse to the outside and receives an ultrasonic echo obtained by reflection on the outside; a transmitting-receiving unit 3 that transmits electrical signals to and receives electrical signals from the ultrasonic probe 2; a processing unit 4 that performs predetermined processing on electrical echo signals which are obtained by means of conversion of the ultrasonic echo; an image processing unit 5 that generates image data corresponding to the electrical echo signals which are obtained by means of conversion of the ultrasonic echo; an input unit 6 that is configured with an interface such as a keyboard, a mouse, or a touch-sensitive panel, and that receives input of a variety of information; a display unit 7 that is configured with a liquid crystal display panel or an organic EL display panel, and that displays a variety of information including the images generated by the image processing unit 5; a memory unit 8 that is used to store a variety of information including the information related to tissue characterizations of known specimens; and a control unit 9 that controls the operations of the ultrasonic diagnosis apparatus 1.

The ultrasonic probe 2 converts electrical pulse signals that are received from the transmitting-receiving unit 3 into ultrasonic pulse (acoustic pulse signals), and includes a signal converting unit 21 for converting the ultrasonic echo that is obtained by reflection from an outside specimen into electrical echo signals. Meanwhile, the ultrasonic probe 2 can be configured to have an ultrasonic transducer performing scanning in a mechanical manner or can be configured to have a plurality of ultrasonic transducers performing scanning in an electronic manner.

The transmitting-receiving unit 3 is electrically connected to the ultrasonic probe 2. With that, the transmitting-receiving unit 3 transmits pulse signals to the ultrasonic probe 2 and receives echo signals representing reception signals from the ultrasonic probe 2. More particularly, based on a predetermined waveform and a predetermined transmission timing, the transmitting-receiving unit 3 generates pulse signals and transmits those pulse signals to the ultrasonic probe 2.

The transmitting-receiving unit 3 includes a signal amplifying unit 31 for amplification of echo signals. With respect to echo signals that are used when the image processing unit 5 generates B-mode image data by converting the amplitude of the echo signals into luminance (hereinafter, referred to as “echo signals for B-mode images”) and with respect to echo signals that are used by the processing unit 4 to perform processing (hereinafter, referred to as “echo signals for processing”), the signal amplifying unit 31 performs amplification with different gains. More particularly, with respect to the echo signals for B-mode images, the signal amplifying unit 31 performs sensitivity time control (STC) in which the gain is directly proportional to the reception depth of the echo signals. In contrast, with respect to the echo signals for processing, the signal amplifying unit 31 performs amplification with a constant gain irrespective of the reception depth. The signal amplifying unit 31 switches between performing amplification of echo signals for B-mode images and performing amplification of echo signals for processing in the units of frames or in the units of lines.

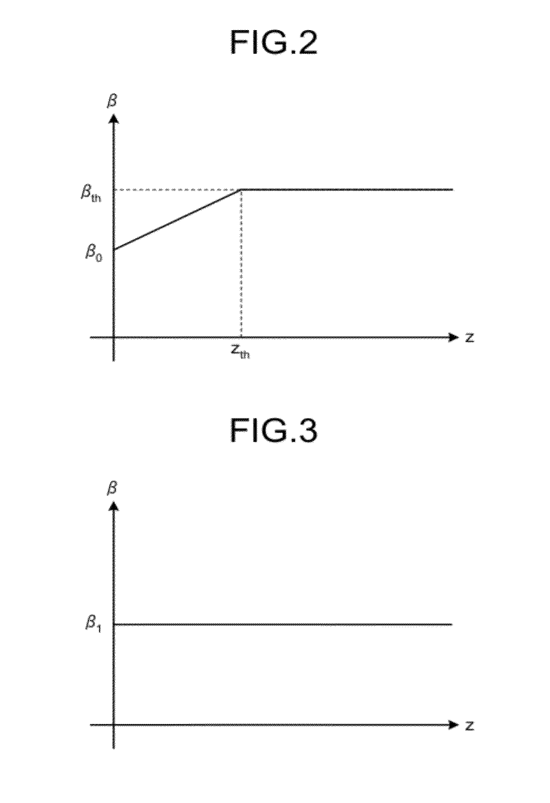

FIG. 2 is a diagram illustrating a relationship between gains and reception depths of echo signals for B-mode images. Herein, with reference to FIG. 2, a reception depth z is calculated based on the elapsed time from the start of receiving ultrasonic sound waves. As illustrated in FIG. 2, when the reception depth z is smaller than a threshold value zₜₕ, a gain β linearly increases from β₀ to βₜₕ accompanying the increase in the reception depth z. When the reception depth z is equal to or greater than the threshold value zₜₕ, the gain β remains constant at βₜₕ. The threshold value zₜₕ is a value at which most of the ultrasonic signals received from a specimen attenuate, and the noise becomes dominant. Meanwhile, more commonly, when the reception depth z is smaller than the threshold value zₜₕ, the gain β may monotonically increase accompanying the increase in the reception depth z.

FIG. 3 is a diagram illustrating a relationship between gains and reception depths of echo signals for processing. With reference to FIG. 3 too, in an identical manner to the case explained with reference to FIG. 2, the reception depth z is calculated based on the elapsed time from the start of receiving ultrasonic sound waves. As illustrated in FIG. 3, with respect to the echo signals for processing, the signal amplifying unit 31 performs amplification with a constant gain β₁ irrespective of the reception depth z.

The transmitting-receiving unit 3 performs operations such as filtering with respect to the echo signals amplified by the signal amplifying unit 31, performs A/D conversion of the echo signals to generate digital RF signals, and outputs those digital RF signals. Meanwhile, when the ultrasonic probe 2 is configured to have a plurality of ultrasonic transducers performing scanning in an electronic manner, the transmitting-receiving unit 3 is configured to include a multichannel circuit for performing beam synthesis corresponding to the ultrasonic transducers.

The processing unit 4 includes a frequency analyzing unit 41 that performs frequency analysis of echo signals by carrying out fast Fourier transformation (FFT) with respect to the digital RF signals that are output by the transmitting-receiving unit 3; includes a feature data extracting unit 42 performs attenuation correction and approximation with respect to the frequency spectrum (power spectrum) calculated by the frequency analyzing unit 41 so that there is a decrease in the contribution of attenuation, which occurs due to the reception depth and the frequency of ultrasonic sound waves being propagated, and extracts feature data of a specimen; and includes a tissue characterization determining unit 43 that determines tissue characterization of a predetermined area of the specimen by referring to the feature data extracted by the feature data extracting unit 42.

The frequency analyzing unit 41 calculates a frequency spectrum with respect to each acoustic ray (line data) by performing FFT with respect to an FFT data group having a predetermined volume of data. Depending on the tissue characterization of a specimen, the frequency spectrum demonstrates a different tendency. That is because of the fact that a frequency spectrum has a correlation with the size, the density, and the acoustic impedance of the specimen that serves as a scatterer which scatters the ultrasonic sound waves.

The feature data extracting unit 42 further includes an approximating unit 421, which performs approximation with respect to the frequency spectrum calculated by the frequency analyzing unit 41 and calculates pre-correction feature data that is the feature data prior to performing attenuation correction; and includes an attenuation correcting unit 422, which extracts feature data by performing attenuation correction with respect to the pre-correction feature data obtained by approximation by the approximating unit 421.

The approximating unit 421 performs linear approximation with respect to the frequency spectrum by means of regression analysis so as to extract pre-correction feature data that characterizes the approximated linear expression. More particularly, by means of regression analysis, the approximating unit 421 calculates a gradient a₀ and an intercept b₀ of the linear expression, as well as calculates the intensity at a specific frequency within the frequency band of the frequency spectrum as the pre-correction feature data. In the first embodiment, it is assumed that, at the central frequency f_{MID}=(f_{LOW}+f_{HIGH})/2, the approximating unit 421 calculates c₀=a₀f_{MID}+b₀ as the intensity (Mid-band fit). However, that is only one example. Herein, the intensity indicates any one parameter of parameters such as voltage, power, acoustic pressure, and acoustic energy.

Of the three components of feature data, the gradient a₀ has a correlation with the size of the scatterer that scatters the ultrasonic sound waves. Generally, it is thought that larger the scatterer, smaller is the value of the gradient. The intercept b₀ has a correlation with the size of the scatterer, the difference in acoustic impedances, and the density (consistency) of the scatterer. More particularly, it is thought that larger the scatterer, greater is the value of the intercept b₀; greater the acoustic impedance, greater is the value of the intercept b₀; and greater the density (concentration) of the scatterer, greater is the value of the intercept b₀. The intensity c₀ at the central frequency f_{MID} (hereinafter, simply referred to as “intensity c₀”) is an indirect parameter derived from the gradient a₀ and the intercept b₀, and represents the spectrum intensity at the center of the valid frequency band. Thus, it is thought that the intensity c₀ has a correlation not only with the size of the scatterer, the difference in acoustic impedances, and the density of the scatterer, but also with the luminosity values of B-mode images to a certain extent. Meanwhile, the approximation polynomial calculated by the feature data extracting unit 42 is not limited to a linear expression. Alternatively, it is also possible to use an approximation polynomial of second-order or more.

The following explanation is given for the correction performed by the attenuation correcting unit 422. An attenuation amount A of ultrasonic sound waves can be expressed as:

A=2αzf (1)

where, α represents the attenuation rate, z represents the reception depth of ultrasonic sound waves, and f represents the frequency. As is clear from Equation (1), the attenuation amount A is proportional to the frequency f. Regarding a living body, the specific value of the attenuation rate α is in the range of 0 to 1.0 (dB/cm/MHz) and desirably is in the range of 0.3 to 0.7 (dB/cm/MHz), and is determined according to the organ to be observed. For example, if the organ to be observed is pancreas, then the attenuation rate α is set to 0.6 (dB/cm/MHz). Meanwhile, in the first embodiment, the configuration can also be such that the value of the attenuation rate α can be modified by an input from the input unit 6.

The attenuation correcting unit 422 corrects the pre-correction feature data (the gradient a₀, the intercept b₀, and the intensity c₀), which has been calculated by the approximating unit 421, in the following manner:

a=a₀=2αz (2)

b=b₀ (3)

c=c₀=2αzf_{MID}(=af_{MID}+b) (4)

As is clear from Equations (2) and (4) too, greater the reception depth of ultrasonic sound waves, greater is the amount of correction during the correction performed by the attenuation correcting unit 422. Meanwhile, with reference to Equation (3), the correction related to the intercept indicates identical transformation. That is because of the fact that the intercept is a frequency component corresponding to the frequency 0 (Hz) and does not get attenuated.

The tissue characterization determining unit 43 calculates, for each set of feature data of the frequency spectrum extracted by the feature data extracting unit 42, the average and the standard deviation of that set of feature data. Then, by referring to the calculated averages and the calculated standard deviations as well as by referring to the averages and the standard deviations of the sets of feature data of the frequency spectrums of known specimens stored in the memory unit 8, the tissue characterization determining unit 43 determines the tissue characterization of a predetermined area of the specimen. Herein, “predetermined area” indicates an area (hereinafter, referred to as “area of concern”) in the image that has been specified by the operator of the ultrasonic diagnosis apparatus 1 upon viewing the images generated by the image processing unit 5. Moreover, for example, “tissue characterization” indicates any one of a cancer, an endocrine tumor, a mucinous tumor, a normal tissue, and a vascular channel. If the specimen is pancreas, then chronic pancreatitis and autoimmune pancreatitis are also considered as tissue characterizations.

The average and the standard deviation of a set of feature data that are calculated by the tissue characterization determining unit 43 reflect the changes at a cellular level such as enlargement or anomaly of the nucleus or reflect the tissue-level changes such as fibrotic growth in the interstitium or substitution of parenchymal tissues with fibers. Thus, depending on the tissue characterization, unique values of the average and the standard deviation are indicated. In consideration of that fact, using the average and the standard deviation of feature data, it becomes possible to correctly determine the tissue characterization of the predetermined area of the specimen.

The image processing unit 5 includes a B-mode image data generating unit 51 that generates B-mode image data from echo signals; and includes a determination-result-display image data generating unit 52 that refers to the data output by the B-mode image data generating unit 51 and by the processing unit 4, and generates determination-result-display image data for displaying the determination result of tissue characterization in the area of concern and for displaying information related to the determination result.

The B-mode image data generating unit 51 generates B-mode image data by performing signal processing on digital signals using a known technology such as bandpass filtering, logarithmic conversion, gain processing, or contrast processing, and by performing data thinning according to the data step width that is decided in accordance to the display range of images in the display unit 7.

The determination-result-display image data generating unit 52 refers to the B-mode image data generated by the B-mode image data generating unit 51, refers to the feature data extracted by the feature data extracting unit 42, and refers to the determination result of the tissue characterization determining unit 43; and generates determination-result-display image data that contains the determination result of tissue characterization in the area of concern and contains a tissue characterization weighted image in which the tissue characterization is highlighted.

The memory unit 8 includes a known-specimen information storing unit 81 that is used to store information on known specimens; includes a gain information storing unit 82 that is used to store information on the gain that is referred to by the signal amplifying unit 31 during amplification; a window function storing unit 83 that is used to store a window function used during frequency analysis performed by the frequency analyzing unit 41; and includes a correction information storing unit 84 that is used to store correction information which is referred to by the attenuation correcting unit 422 while performing operations.

The known-specimen information storing unit 81 is used to store the feature data of frequency spectrums extracted for known specimens and the tissue characterizations of those known specimens in a corresponding manner. In addition, with respect to feature data of the frequency spectrum related to a known specimen; the average and the standard deviation calculated for each group, which is classified on the basis of the tissue characterization of that known specimen, as well as all feature data of that known specimen is stored in the known-specimen information storing unit 81. Herein, it is assumed that the feature data of a known specimen is extracted by performing an identical operation to that performed in the first embodiment. However, the feature data extracting operation for a known specimen need not be performed in the ultrasonic diagnosis apparatus 1. Meanwhile, it is desirable that the information on known specimens stored in the known-specimen information storing unit 81 is reliable in nature regarding tissue characterizations. The gain information storing unit 82 is used to store the relationship between gains and reception depths illustrated in FIG. 2 and FIG. 3. The window function storing unit 83 is used to store at least one window function of the window functions such as Hamming, Hanning, and Blackman. The correction information storing unit 84 is used to store the information related to the conversion of Equations (2) to (4).

Meanwhile, the memory unit 8 is put into practice with a ROM, which is used to store in advance operating programs of the ultrasonic diagnosis apparatus 1 according to the first embodiment and to store programs for running a predetermined OS; and with a RAM, which is used to store operating parameters and data of each operation.

In the ultrasonic diagnosis apparatus 1 having the abovementioned functional configuration, the constituent elements other than the ultrasonic probe 2 are put into practice with a computer that includes a CPU for performing processing and control. The CPU in the ultrasonic diagnosis apparatus 1 reads, from the memory unit 8, the information and various programs including the operating programs of the ultrasonic diagnosis apparatus 1; and performs processing related to the operation method of the ultrasonic diagnosis apparatus 1 according to the first embodiment.

The operating programs of the ultrasonic diagnosis apparatus 1 according to the first embodiment can also be recorded in a computer readable recording medium such as a hard disk, a flash memory, a CD-ROM, a DVD-ROM, or a flexible disk for the purpose of distribution.

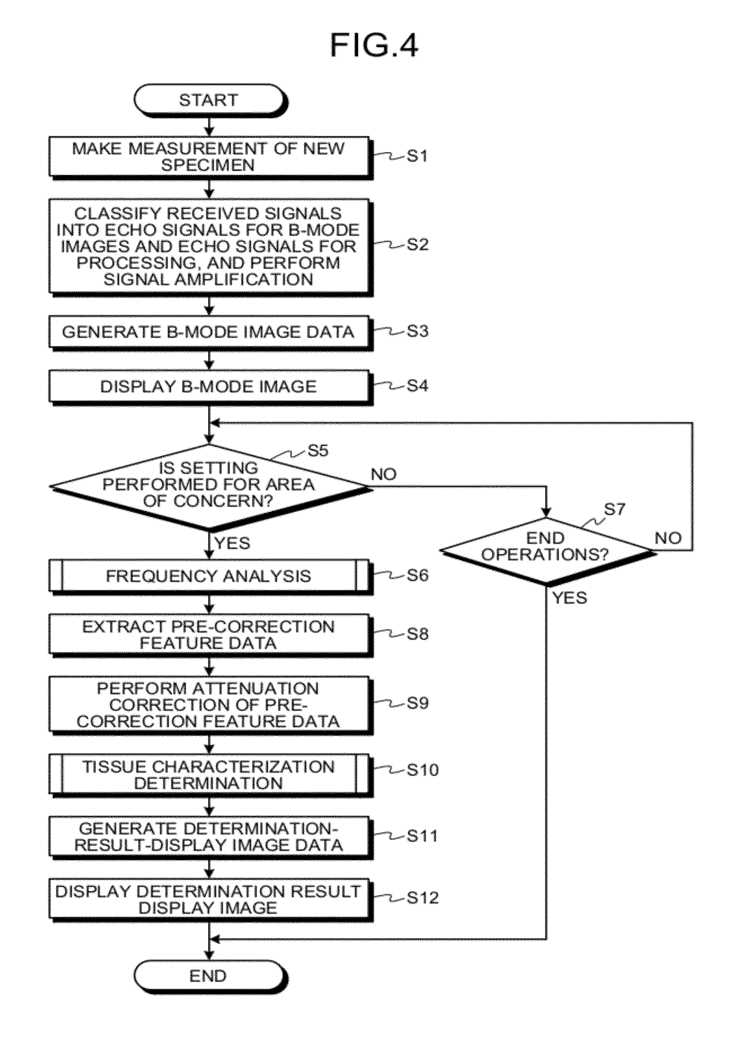

FIG. 4 is a flowchart explaining an overview of the operations performed by the ultrasonic diagnosis apparatus 1 having the configuration explained above. With reference to FIG. 4, firstly, the ultrasonic diagnosis apparatus 1 makes a measurement of a new specimen using the ultrasonic probe 2 (Step S1).

Then, the signal amplifying unit 31 receives echo signals from the ultrasonic probe 2 and performs amplification by classifying the received echo signals into echo signals for B-mode images and echo signals for processing (Step S2). Herein, the signal amplifying unit 31 performs amplification based on the relationship between gains and reception depths as illustrated in FIG. 2 and FIG. 3. Meanwhile, the timing of performing amplification of echo signals for B-mode images and the timing of performing amplification of echo signals for processing can be switched in the units of frames or in the units of lines.

Subsequently, the B-mode image data generating unit 51 generates B-mode image data using the echo signals for B-mode images output by the transmitting-receiving unit 3 (Step S3).

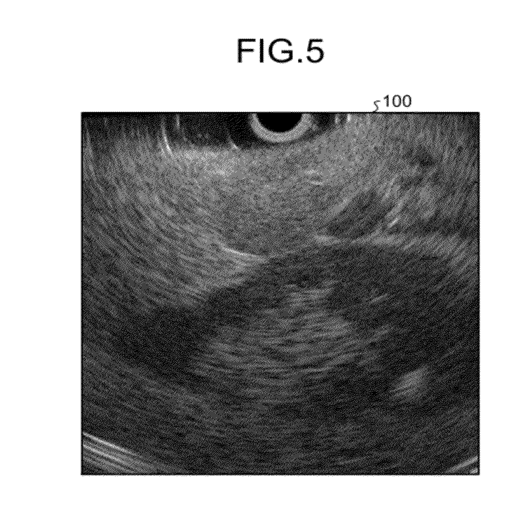

Then, the control unit 9 performs control so that the display unit 7 displays a B-mode image corresponding to the B-mode image data generated by the B-mode image data generating unit 51 (Step S4). FIG. 5 is a diagram illustrating an example of a B-mode image displayed by the display unit 7. A B-mode image 100 illustrated in FIG. 5 is a grayscale image in which variables R (red), G (green), and B (blue), which are variables when the RGB color system is adopted as the color space, have identical values.

Subsequently, if settings for the area of concern are performed via the input unit 6 (Yes at Step S5), the frequency analyzing unit 41 performs frequency analysis by means of FFT and calculates a frequency spectrum (Step S6). At Step S6, it is also possible to set the entire area of an image as the area of concern. Meanwhile, if settings for the area of concern are not yet performed (No at Step S5) but if an instruction to end operations is input via the input unit 6 (Yes at Step S7); then the ultrasonic diagnosis apparatus 1 ends the operations. In contrast, neither settings for the area of concern are performed (No at Step S5) nor an instruction to end operations is input via the input unit 6 (No at Step S7), then the system control returns to Step S5.

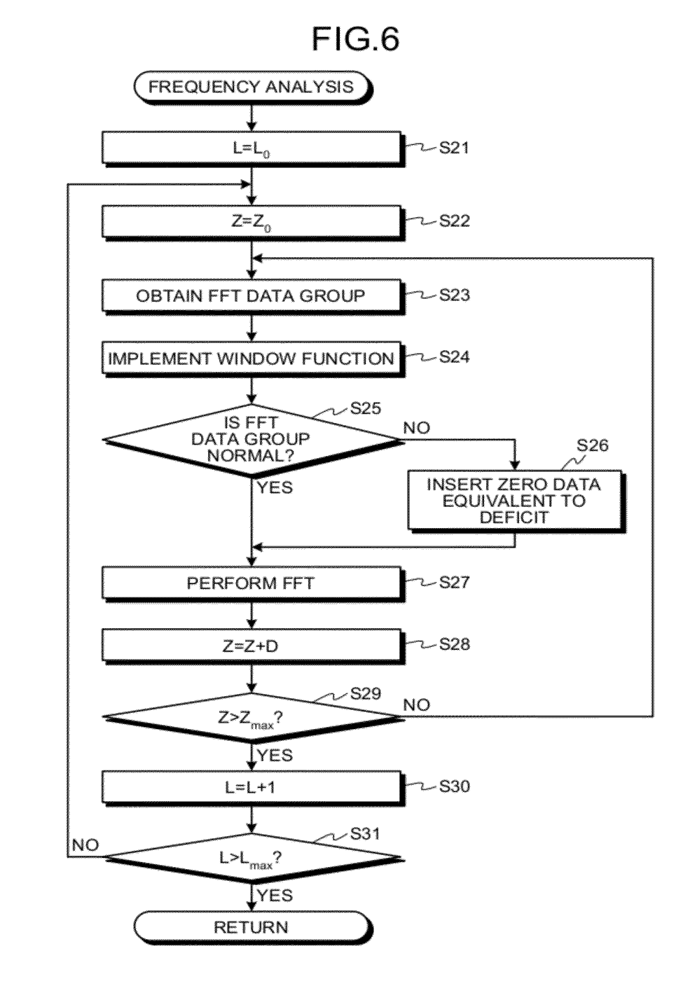

Herein, the operation performed by the frequency analyzing unit 41 at Step S6 is explained in detail with reference to a flowchart illustrated in FIG. 6. Firstly, the frequency analyzing unit 41 sets an acoustic ray number L of the acoustic ray to be initially analyzed to an initial value L₀ (Step S21). The initial value L₀ can be assigned, for example, to the acoustic ray received at the start by the transmitting-receiving unit 3 or to the acoustic ray corresponding to the border position on any one of the left and right sides of the area of concern set via the input unit 6.

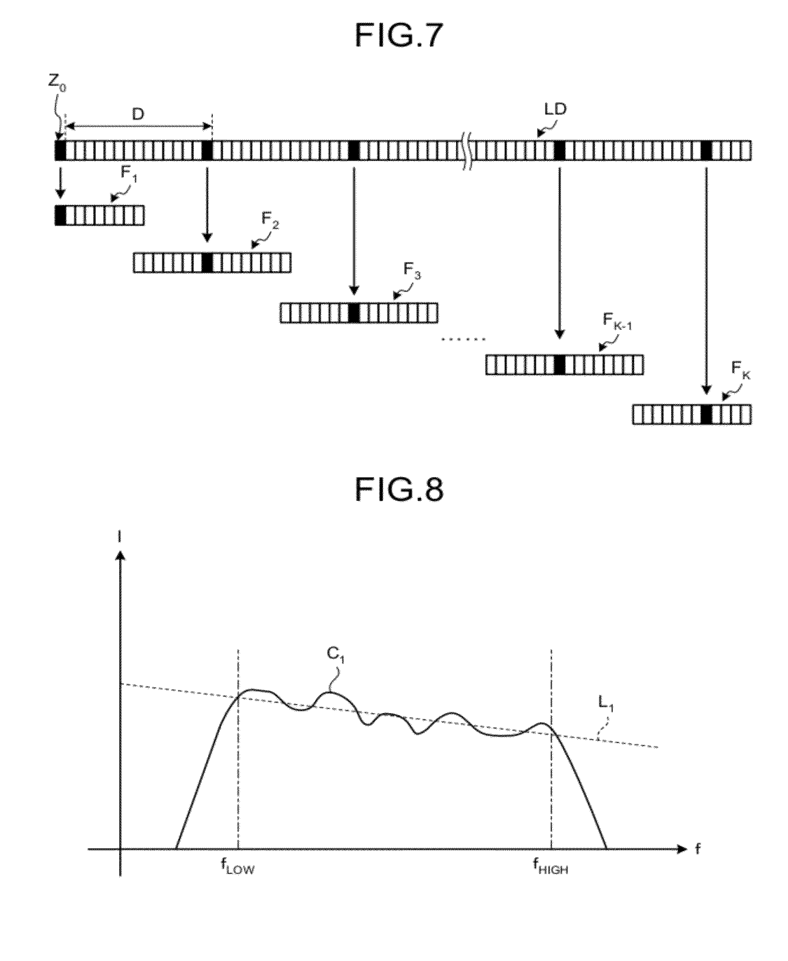

Then, the frequency analyzing unit 41 calculates the frequency spectrum of all of a plurality of data positions set on a single acoustic ray. Regarding that, firstly, the frequency analyzing unit 41 sets an initial value Z₀ of a data position Z (equivalent to reception depth) that is representative of a sequence of data groups (FFT data groups) obtained for the purpose of FFT (Step S22). FIG. 7 is a diagram that schematically illustrates data arrangement of a single acoustic ray. In an acoustic ray LD illustrated in FIG. 7, a white rectangle or a black rectangle represents a single set of data. The acoustic ray LD is discretized by time intervals corresponding to the sampling frequency (such as 50 MHz) used during A/D conversion performed by the transmitting-receiving unit 3. In FIG. 7, it is illustrated that the first set of data on the acoustic ray LD is set as the initial value Z₀ of the data position Z. Meanwhile, FIG. 7 is only an example, and the position of the initial value Z₀ can be set in an arbitrary manner. For example, the data position Z corresponding to the position at the top edge of the area of concern can be set as the initial value Z₀.

Then, the frequency analyzing unit 41 obtains the FFT data group at the data position Z (Step S23) and implements the window function, which is stored in the window function storing unit 83, to the FFT data group that has been obtained (Step S24). By implementing the window function to the FFT data group, it becomes possible to avoid discontinuity at the borders in the FFT data group. As a result, artifacts can be prevented from occurring.

Subsequently, the frequency analyzing unit 41 determines whether or not the FFT data group at the data position Z is a normal data group (Step S25). Herein, it is necessary that the number of sets of data in an FFT data group is in power-of-two. In the following explanation, it is assumed that the number of sets of data in the FFT data group is 2ⁿ (where n is a positive integer). When an FFT data group is normal, it means that the data position Z is the 2ⁿ⁻¹-th position from the front of the FFT data group. In other words, when an FFT data group is normal, it means that there are 2ⁿ⁻¹−1 (=N) number of sets of data prior to the data position Z, and there are 2ⁿ⁻¹ (=M) number of sets of data subsequent to the data position Z. In the example illustrated in FIG. 7, FFT data groups F₂, F₃, and F_{K-1} are normal data groups; while FFT data groups F₁ and F_{K} are abnormal data groups. However, in FIG. 7, it is assumed that n=4 (N=7, M=8).

If the determination result of Step S25 indicates that the FFT data group at the data position Z is normal (Yes at Step S25), then the system control proceeds to Step S27 (described later).

If the determination result of Step S25 indicates that the FFT data group at the data position Z is not normal (No at Step S25), then the frequency analyzing unit 41 inserts zero data equivalent to the deficit and generates a normal FFT data group (Step S26). To the FFT data group that is determined to be not normal at Step S25, the window function is implemented prior to the addition of zero data. Hence, even if zero data is inserted, discontinuity in data does not occur. Once the operation at Step S26 is completed, the system control proceeds to Step S27.

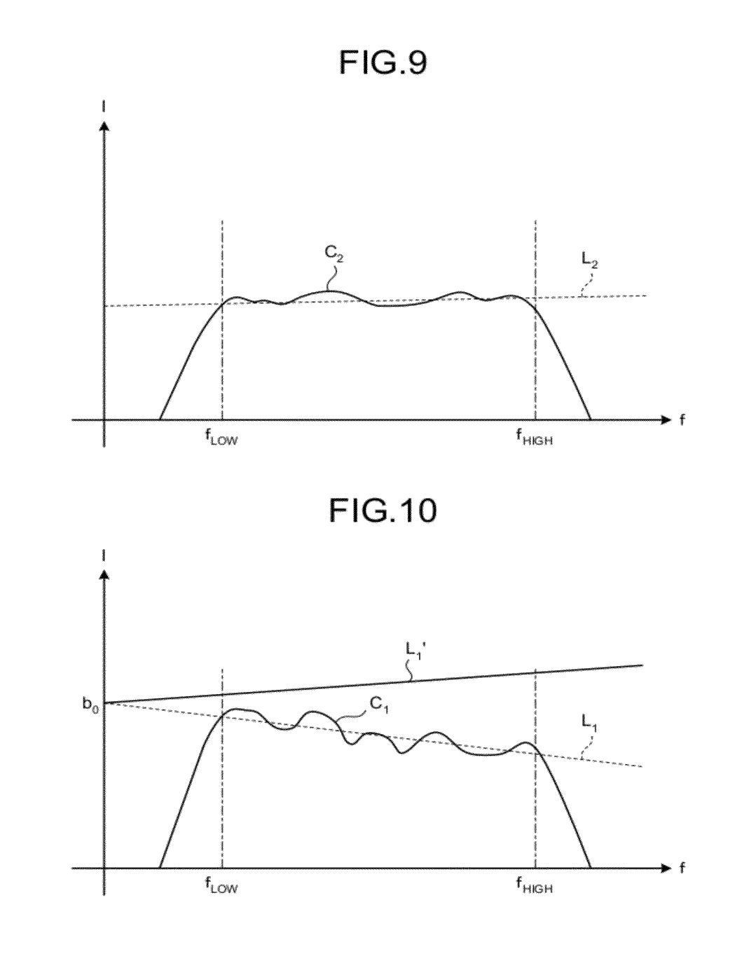

At Step S27, the frequency analyzing unit 41 performs FFT using the FFT data groups and obtains the frequency spectrum (Step S27). FIG. 8 and FIG. 9 are diagrams illustrating examples of the frequency spectrum calculated by the frequency analyzing unit 41. In FIG. 8 and FIG. 9, the horizontal axis f represents the frequency and the vertical axis I represents the intensity. In frequency spectrum curves C₁ and C₂ illustrated in FIG. 8 and FIG. 9, respectively; a lower limit frequency f_{LOW} and a high limit frequency f_{HIGH} of the frequency spectrum are parameters determined on the basis of the frequency band of the ultrasonic probe 2 and the frequency band of the pulse signals transmitted by the transmitting-receiving unit 3. For example, f_{LOW} is equal to 3 MHz and f_{HIGH} is equal to 10 MHz. Meanwhile, regarding a straight line L₁ illustrated in FIG. 8 and a straight line L₂ illustrated in FIG. 9, the explanation is given later while explaining the feature data extracting operation. In the first embodiment, curve lines and straight lines are formed of sets of discreet points. The same is the case in other embodiments described later.

Subsequently, the frequency analyzing unit 41 adds a predetermined data step width D to the data position Z, and calculates the data position Z at the FFT data group to be analyzed next (Step S28). Herein, it is desirable that the data step width D is matched with the data step width used at the time when the B-mode image data generating unit 51 generates B-mode image data. However, when the object is to reduce the amount of operations in the frequency analyzing unit 41, it is also possible to set the data step width D to a larger value than the data step width used by the B-mode image data generating unit 51. In FIG. 7, it is illustrated that D=15.

Subsequently, the frequency analyzing unit 41 determines whether or not the data position Z is greater than a final data position Zₘₐₓ (Step S29). Herein, the final data position Zₘₐₓ can be set to the data length of the acoustic ray LD or to the data position corresponding to the lower edge of the area of concern. If the determination result indicates that the data position Z is greater than the final data position Zₘₐₓ (Yes at Step S29), then the frequency analyzing unit 41 increments the acoustic ray number L by 1 (Step S30). On the other hand, if the determination result indicates that the data position Z is equal to or smaller than the final data position Zₘₐₓ (No at Step S29), then the system control returns to Step S23. In this way, with respect to a single acoustic ray LD, the frequency analyzing unit 41 performs FFT for [{(Zₘₐₓ−Z₀)/D}+1] (=K) number of FFT data groups. Herein, [X] represents the largest integer not exceeding X.

If the acoustic number L that has been incremented at Step S30 is greater than a final acoustic number L. (Yes at Step S31), then the system control returns to the main routine illustrated in FIG. 4. On the other hand, if the acoustic number L that has been incremented at Step S30 is equal to or smaller than the final acoustic number Lₘₐₓ (No at Step S31), then the system control returns to Step S22.

In this way, the frequency analyzing unit 41 performs FFT for K number of times with respect to each of (Lₘₐₓ−L₀+1) number of acoustic rays. For example, the final acoustic ray number Lₘₐₓ can be assigned to the final acoustic ray received by the transmitting-receiving unit 3 or to the acoustic ray corresponding to the border position on any one of the left and right sides of the area of concern. In the following explanation, the total number of times for which the frequency analyzing unit 41 performs FFT with respect to all acoustic rays is (Lₘₐₓ−L₀+1)×K and is referred to as “P”.

Subsequent to the frequency analyzing operation performed at Step S6 as described above, the approximating unit 421 performs, as an approximation operation, regression analysis of the P number of frequency spectrums calculated by the frequency analyzing unit 41 and extracts the pre-correction feature data (Step S8). More particularly, the approximating unit 421 performs regression analysis and calculates the linear expression for approximation of the frequency spectrums in the frequency band of f_{LOW}<f<f_{HIGH}; and then calculates the gradient a₀, the intercept b₀, and the intensity c₀, which characterize the linear expression, as the pre-correction feature data. The straight line L₁ illustrated in FIG. 8 and the straight line L₂ illustrated in FIG. 9 are regression lines obtained by performing regression analysis of the frequency spectrum curve C₁ and the frequency spectrum curve C₂, respectively, at Step S8.

Then, the attenuation correcting unit 422 performs attenuation correction of the pre-correction feature data extracted by the approximating unit 421 (Step S9). For example, when the data sampling frequency is 50 MHz, the time interval for data sampling is 20 (nsec). If the velocity of sound is assumed to be 1530 (m/sec), then the spacing among data sampling is equal to 1530 (m/sex)×20 (nsec)/2=0.0153 (mm). If “k” is assumed to be the number of data steps from the first set of data of the acoustic ray LD up to the data position of the FFT data group to be processed, then the data position Z thereof is equal to 0.0153 k (mm). The attenuation correcting unit 422 substitutes the value of the data position Z, which is obtained in the manner described above, in the reception depth z specified in Equations (2) to (4) mentioned above, and calculates the gradient a, the intercept b, and the intensity c. FIG. 10 is a diagram illustrating a straight line that is determined from the feature data obtained upon performing attenuation correction of the feature data related to the straight line L₁ illustrated in FIG. 8. A straight line L₁′ illustrated in FIG. 10 can be expressed as:

I=af+b=(a₀+2αZ)f+b₀ (5)

As is clear from Equation (5), as compared to the straight line L₁, the straight line L₁′ has a greater gradient with the same intercept value.

Subsequently, based on the feature data extracted by the feature data extracting unit 42 and based on the information on known specimens stored in the known-specimen information storing unit 81, the tissue characterization determining unit 43 determines the tissue characterization of the area of concern of the specimen (Step S10).

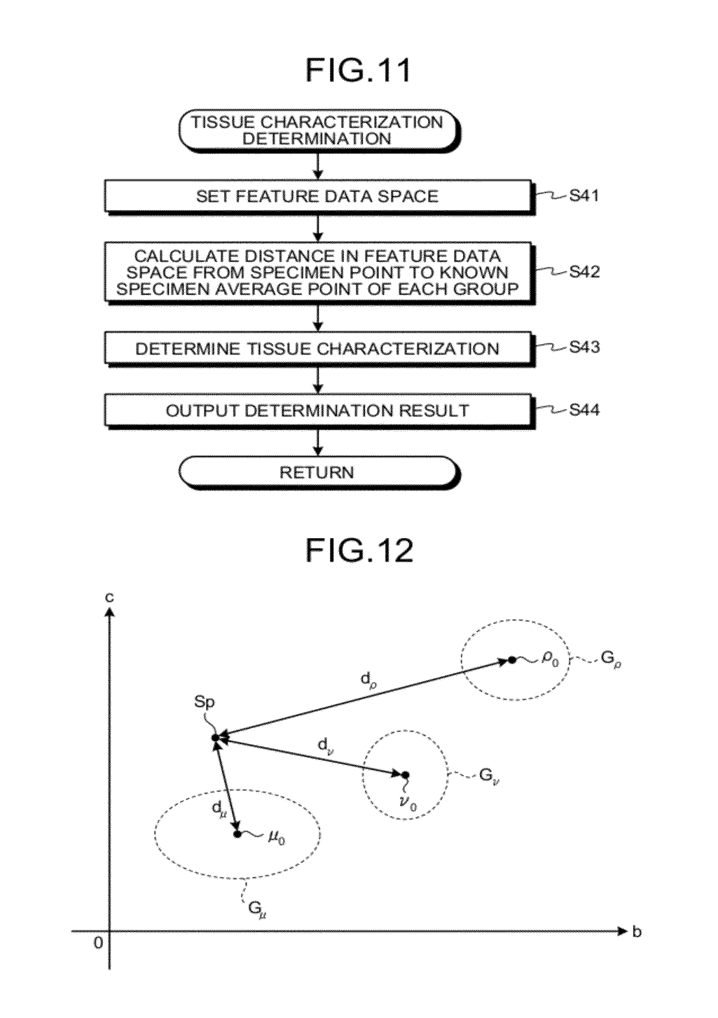

Herein, the operation performed by the tissue characterization determining unit 43 at Step S10 is explained in detail by referring to a flowchart illustrated in FIG. 11. Firstly, the tissue characterization determining unit 43 sets a feature data space that is to be used while determining the tissue characterization (Step S41). In the first embodiment, among the gradient a, the intercept b, and the intensity c that are the three components of feature data; there are two independent parameters. Thus, a two-dimensional space can be set that has any two of the three components of feature data as the components. Alternatively, a one-dimensional space can also be set that has any one of the three components of feature data as the component. At Step S41, it is assumed that the feature data space to be set is determined in advance. However, alternatively, the operator can be allowed to set the desired feature data space using the input unit 6.

FIG. 12 is a diagram illustrating an example of the feature data space set by the tissue characterization determining unit 43. In the feature data space illustrated in FIG. 12, the horizontal axis represents the intercept b and the vertical axis represents the intensity c. In FIG. 12, a point Sp represents the point (hereinafter, referred to as “specimen point Sp”) that has the intercept b and the intensity c calculated regarding the target specimen for determination as the coordinates of the feature data space. Moreover, areas G_{μ}, G_{ν}, and G_{ρ} illustrated in FIG. 12 represent groups in which known specimens stored in the known-specimen information storing unit 81 have tissue characterizations of μ, ν, and ρ, respectively. In the example illustrated in FIG. 12, in the feature data space, the three groups G_{μ}, G_{ν}, and G_{ρ} are present in mutually exclusive areas.

In the first embodiment, even while obtaining the feature data of a known specimen; the tissue characterizations are classified and determined with the feature data, which is obtained by performing attenuation correction with respect to the pre-correction feature data of the frequency spectrum obtained by means of frequency analysis, serving as the index. Hence, it becomes possible to make distinction between mutually different tissue characterizations. Particularly, in the first embodiment, the feature data that has been subjected to attenuation correction is used. Therefore, as compared to the case of using the feature data that is extracted without performing attenuation correction, the area of each tissue characterization in the feature data space can be obtained in a more distinctly separated state.

Subsequent to Step S41, the tissue characterization determining unit 43 calculates distances d_{μ}, d_{ν}, and d_{ρ} from the specimen point Sp to points μ₀, ν₀, and ρ₀, respectively, (hereinafter, these points are referred to as “known specimen average points”); where the points μ₀, ν₀, and ρ₀ have the average of the intercept b and the average of the intensity c of the frequency spectrum of the FFT data group included in the groups G_{μ}, G_{ν}, and G_{ρ}, respectively, as the coordinates in the feature data space (Step S42). Meanwhile, if the b-axis component and the c-axis component in the feature data space differ in scale by a large extent, it is desirable to appropriately perform weighting so that each distance contributes in a substantially equal manner.

Then, based on the distances calculated at Step S42, the tissue characterization determining unit 43 determines the tissue characterizations of all specimen points including the specimen point Sp (Step S43). For example, in the case illustrated in FIG. 12, since the distance d_{μ}is the smallest, the tissue characterization determining unit 43 determines that μ is the tissue characterization of the specimen. Meanwhile, if the specimen point Sp is extremely separated from the known specimen average points μ₀, ν₀, and ρ₀; then the determination result of tissue characterizations is low in terms of reliability even if the smallest values of the distances d_{μ}, d_{ν}, and d_{ρ} are obtained. In that regard, when the distances d_{μ}, d_{ν}, and d_{ρ} are greater than a predetermined threshold value, the tissue characterization determining unit 43 can be configured to output an error signal. Moreover, when that smallest value of the distances d_{μ}, d_{ν}, and d_{ρ} is obtained for two or more times; the tissue characterization determining unit 43 can be configured to select all tissue characterizations corresponding to the smallest value or to select only one tissue characterization according to predetermined rules. In the latter case, for example, a method can be implemented in which a high-grade tissue characterization such as cancer is set to have a high priority. Meanwhile, alternatively, when that smallest value of the distances d_{μ}, d_{ν}, and d_{ρ} is obtained for two or more times; the tissue characterization determining unit 43 can be configured to output an error signal.

Then, the tissue characterization determining unit 43 outputs the distance calculation result obtained at Step S42 and the determination result obtained at Step S43 (Step S44). That marks the end of the tissue characterization determining operation performed at Step S10.

Subsequent to Step S10 described above, the determination-result-display image data generating unit 52 generates determination-result-display image data by referring to the B-mode image data output by the B-mode image data generating unit 51, the feature data calculated by the feature data extracting unit 42, and the determination result obtained by the tissue characterization determining unit 43 (Step S11).

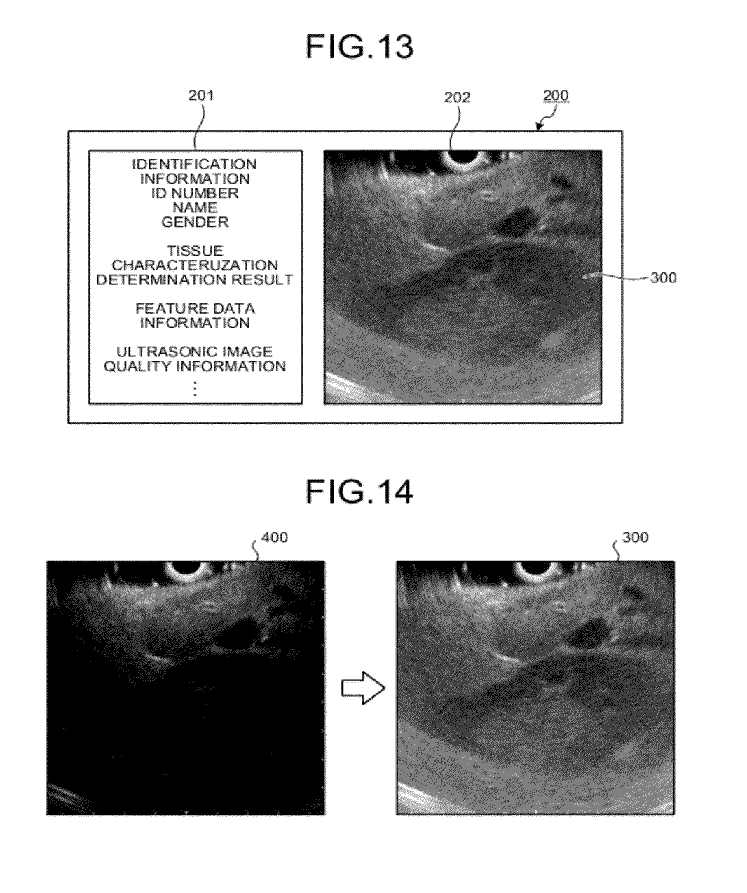

Then, the display unit 7 displays a determination result display image generated by the determination-result-display image data generating unit 52 (Step S12). FIG. 13 is a diagram illustrating a display example of the determination result display image displayed by the display unit 7. A determination result display image 200 illustrated in FIG. 13 includes an information displaying portion 201, which is used for displaying a variety of related information including the tissue characterization determination result, and an image displaying portion 202, which is used for displaying the tissue characterization weighted image in which the tissue characterization is highlighted based on the B-mode image.

In the information displaying portion 201, for example, following information is displayed: identification information (ID number, name, gender) of a specimen; the tissue characterization determination result obtained by the tissue characterization determining unit 43; feature data information used in performing tissue characterization determination; and ultrasonic image quality information such as gain and contrast. Herein, as the feature data information, the display can be performed using the average and the standard deviation of feature data of the frequency spectrums of Q number of FFT data groups present inside the area of concern. More particularly, in the information displaying portion 201, for example, it is possible to display the following information: gradient=1.5±0.3 (dB/MHz); intercept=−60±2 (dB); and intensity=−50±1.5 (dB).

As compared to the B-mode image 100 illustrated in FIG. 5, a tissue characterization weighted image 300 displayed in the image displaying portion 202 is a grayscale image in which the intercept b is evenly assigned among R (red), G (green), and B (blue).

When the display unit 7 displays the determination result display image 200 having the abovementioned configuration, the operator can correctly understand the tissue characterization of the area of concern. However, determination result display images are not limited to the abovementioned configuration. Alternatively, for example, as a determination result display image, it is possible to display side-by-side a tissue characterization weighted image and a B-mode image. With that, the differences in the two images become recognizable on the same screen.

FIG. 14 is a diagram explaining the effect of attenuation correction performed by the ultrasonic diagnosis apparatus 1 according to the first embodiment. An image 400 illustrated in FIG. 14 is a tissue characterization weighted image not subjected to attenuation correction. In the tissue characterization weighted image 400, in the area having a large reception depth (the lower area in FIG. 14), the signal intensity decreases due to the effect of attenuation, thereby making the image darker. In contrast, regarding the tissue characterization weighted image 300 for which attenuation correction is performed, it can be seen that the image has got a uniform brightness throughout the screen.

As described above, according to the first embodiment of the present invention, approximation is performed with respect to a frequency spectrum that has been obtained by analyzing the frequencies of received ultrasonic sound waves. Then, the feature data of a specimen is extracted by performing attenuation correction by which there is a decrease in the contribution of attenuation, which occurs due to the reception depth and the frequency of the ultrasonic sound waves. Based on the extracted feature data of the specimen and based on the feature data of a plurality of known specimens, the tissue characterization of a predetermined area of the specimen is determined. Hence, without having to make use of the strain amount or the degree of elasticity of the body tissues, it becomes possible to make clear distinction between different tissues. As a result, tissue characterizations can be distinguished with accuracy and the measurement result can be enhanced in terms of reliability.

Moreover, according to the first embodiment, since attenuation correction is performed with respect to the extracted feature data, it becomes possible to eliminate the effect of attenuation that occurs during the propagation of ultrasonic sound waves. That makes it possible to determine tissue characterizations with a higher degree of accuracy.

Meanwhile, in the first embodiment, the tissue characterization weighted image 300 described above is only an example. Alternatively, for example, it is also possible to display a tissue characterization weighted image in the form of a color image by assigning R (red), G (green), and B (blue) serving as visual information to each of the gradient a, the intercept, and the intensity c that are the three components of feature data. In this case, in a tissue characterization weighted image, colors are displayed corresponding to tissue characterizations. Hence, based on the color distribution in the image, the operator can understand the tissue characterization of the area of concern. Explained below is a specific example of using a color image.

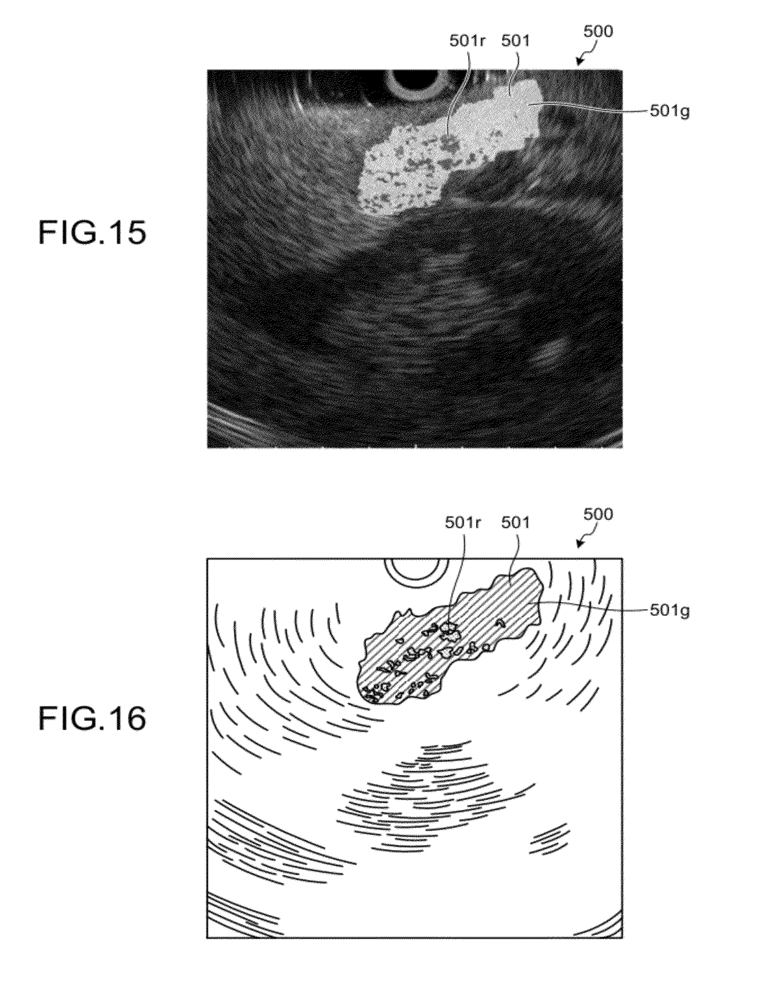

FIG. 15 is a diagram illustrating a display example (first example) of a tissue characterization weighted image in which a color image is used. FIG. 16 is a diagram that schematically illustrates a black-and-white image of the image illustrated in FIG. 15. In a tissue characterization weighted image 500 illustrated in FIG. 15 and FIG. 16, only a specific area 501 is displayed as a color image, and the remaining area is displayed without modification as a B-mode image. The specific area 501 is broadly divided into a greenish area 501g and a reddish area 501r, with the boundary portion between those two areas displayed in a yellowish color (not illustrated in FIG. 16). As illustrated in FIG. 15, it is not the case that each area is made of only a single color. For example, the greenish area 501g is an area including pixels having colors close to the green color. Similarly, the reddish area 501r is an area including pixels having colors close to the red color.

The determination-result-display image data generating unit 52 converts the pixel values of the B-mode image, which corresponds to the specimen point included in a predetermined group in the feature data space, into pixel values colored according to the feature data; and generates the tissue characterization weighted image 500 in which the area 501 is displayed as a color image. With that tissue characterization weighted image 500, it becomes possible to highlight the area of a predetermined tissue characterization as a color image. Hence, the person in charge of diagnosis can easily understand the area in which specific tissues are present. That enables achieving enhancement in the detection rate.

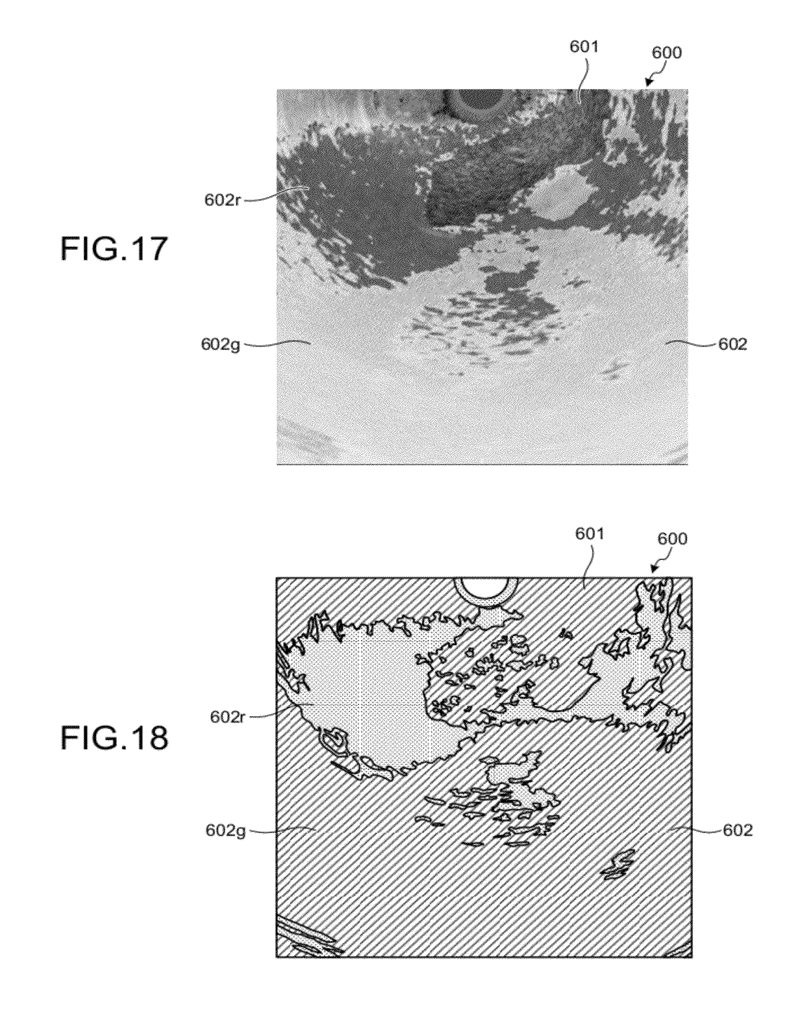

FIG. 17 is a diagram illustrating a display example (second example) of a tissue characterization weighted image in which a color image is used. FIG. 18 is a diagram that schematically illustrates a black-and-white image of the image illustrated in FIG. 17. A tissue characterization weighted image 600 illustrated in FIG. 17 and FIG. 18 is an image generated using the same B-mode image as used in the tissue characterization weighted image 500 illustrated in FIG. 15 and FIG. 16. However, in the tissue characterization weighted image 600; an area 601, which corresponds to the area 501, is displayed without modification as the B-mode image, and a remaining area 602 is displayed as a color image. More particularly, the area 602 is broadly divided into a greenish area 602g and a reddish area 602r, with the boundary portion between those two areas displayed in a yellowish color (not illustrated in FIG. 18). In FIG. 18 too, in an identical manner to FIG. 16, the greenish area 602g is an area including pixels having colors close to the green color; while the reddish area 602r is an area including pixels having colors close to the red color.

The determination-result-display image data generating unit 52 converts the pixel values of the B-mode image, which corresponds to the specimen point included in a predetermined group in the feature data space, into pixel values colored according to the feature data; and generates the tissue characterization weighted image 600 in which the area other than the area 601 is displayed as a color image. With that tissue characterization weighted image 600, it becomes possible to display the area of a predetermined tissue characterization as the B-mode image and to display the remaining area as a color image. Hence, the person in charge of diagnosis can easily understand the area in which specific tissues are present. That enables achieving enhancement in the detection rate. Besides, the internal structure of those tissues can be correctly understood based on the B-mode image.

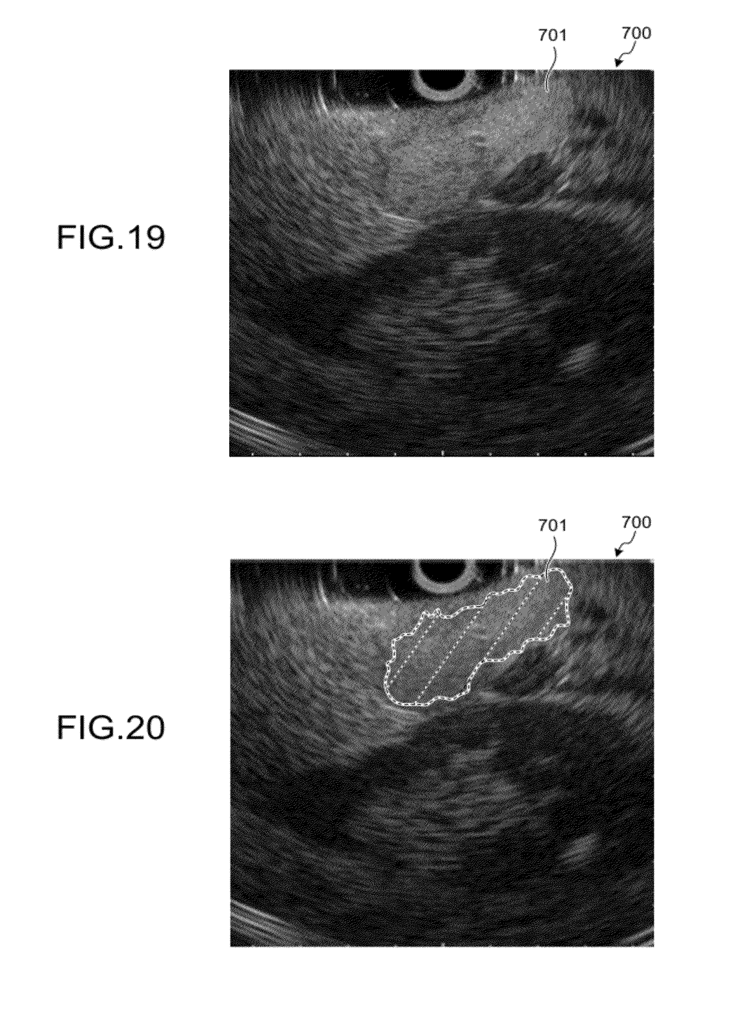

FIG. 19 is a diagram illustrating a display example (third example) of a tissue characterization weighted image in which a color image is used. FIG. 20 is a diagram that schematically illustrates a black-and-white image of the image illustrated in FIG. 19. A tissue characterization weighted image 700 illustrated in FIG. 19 and FIG. 20 is an image generated using the same B-mode image as used in the tissue characterization weighted image 500 illustrated in FIG. 15 and FIG. 16. However, in the tissue characterization weighted image 700; it is not only that an area 701, which corresponds to the area 501, is displayed a color image but also that the display enables to understand the internal structure of the area 701. The determination-result-display image data generating unit 52 performs an operation in which the pixels corresponding to the specimen point included in a predetermined group in the feature data space have new pixel values obtained by weighting the pixel values of the B-mode image and the pixel values of the color image that are determined depending on the tissue characterization, and by taking an average of the weighted pixels values; and generates the tissue characterization weighted image 700 that has the area 701 in which the B-mode image and the color image are displayed in a superposed manner. With that tissue characterization weighted image 700, it becomes possible to display only the area 701 having a predetermined tissue characterization as a color image. Hence, the person in charge of diagnosis can easily understand the area in which specific tissues are present. That enables achieving enhancement in the detection rate. Besides, the internal structure of those tissues can be correctly understood by referring to the information of the B-mode image.

Meanwhile, instead of forming the color space with the RGB color system; it is also possible to form the color space with variables such as cyan, magenta, and yellow of a complementary color system, and to assign feature data to each variable.

### Second Embodiment

In a second embodiment of the present invention, the feature data extracting operation performed by a feature data extracting unit is different than the first embodiment. The configuration of an ultrasonic diagnosis apparatus according to the second embodiment is same as the configuration of the ultrasonic diagnosis apparatus 1 according to the first embodiment. Thus, in the following explanation, the constituent elements identical to those in the ultrasonic diagnosis apparatus 1 are referred to by the same reference numerals.

During the feature data extracting operation according to the second embodiment, firstly, the attenuation correcting unit 422 performs attenuation correction with respect to the frequency spectrum calculated by the frequency analyzing unit 41. Then, the approximating unit 421 performs approximation with respect to the frequency spectrum that has been subjected to attenuation correction by the attenuation correcting unit 422, and extracts the feature data of the frequency spectrum.

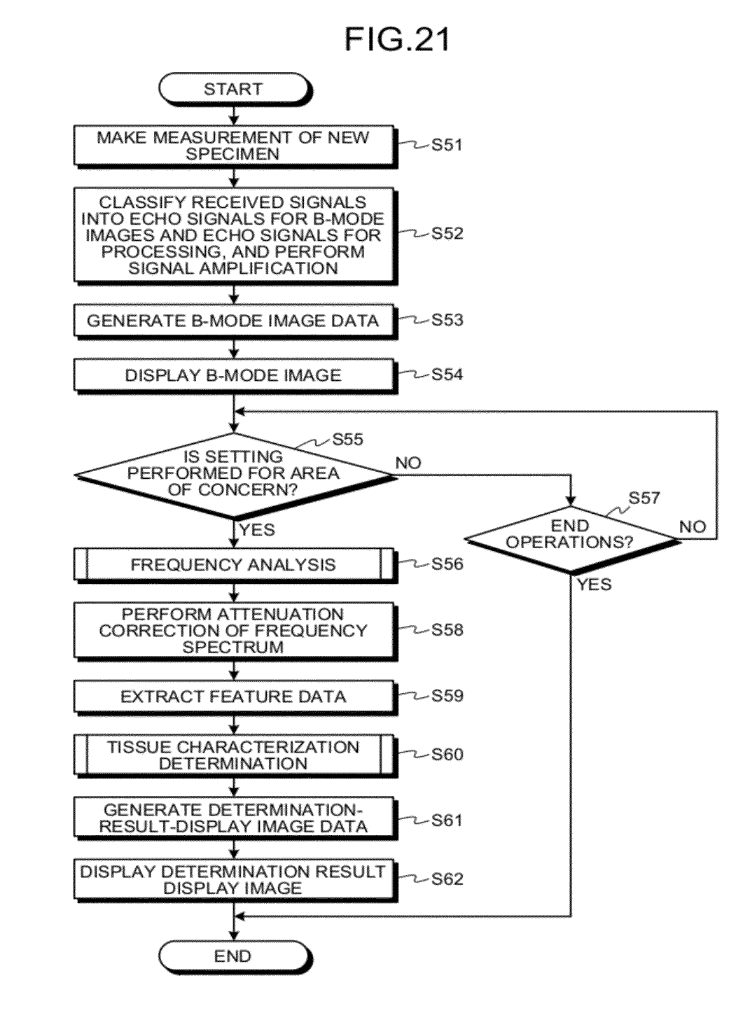

FIG. 21 is a flowchart explaining an overview of the operations performed by the ultrasonic diagnosis apparatus 1 according to the second embodiment. With reference to FIG. 21, the operations performed at Step S51 to Step S57 are respectively identical to the operations performed at Step S1 to Step S7 illustrated in FIG. 4.

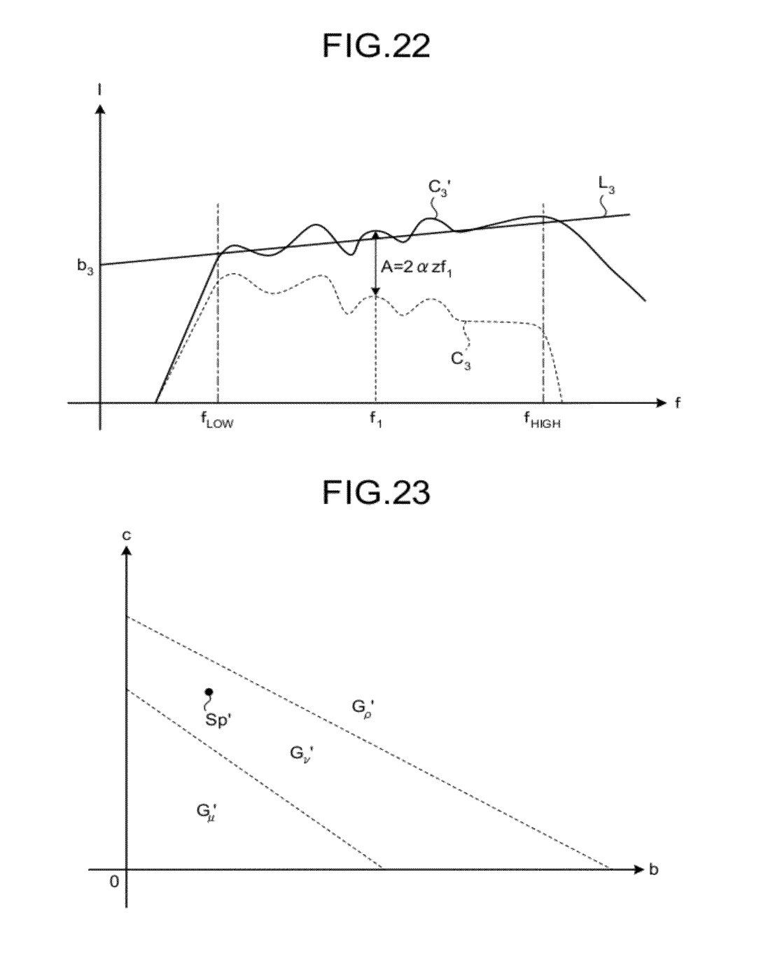

At Step S58, the attenuation correcting unit 422 performs attenuation correction with respect to all frequency spectrums that are calculated by the frequency analyzing unit 41 by means of FFT (Step S58). FIG. 22 is a diagram that schematically illustrates an overview of the operation performed at Step S58. As illustrated in FIG. 22, with respect to a frequency spectrum curve C₃, the attenuation correcting unit 422 performs correction in the form of adding the attenuation amount A given in Equation (1) to the intensity I for all frequencies f, and obtains a new frequency spectrum curve C₃′. As a result, it becomes possible to obtain a frequency spectrum in which the contribution of attenuation occurring due to the propagation of ultrasonic sound waves is reduced.

Subsequently, the approximating unit 421 performs regression analysis of all frequency spectrums that are subjected to attenuation correction by the attenuation correcting unit 422, and extracts the feature data of the frequency spectrums (Step S59). More particularly, the approximating unit 421 performs regression analysis and calculates the gradient a, the intercept b, and the intensity c at the central frequency f_{MID}, which characterize the linear expression. A straight line L₃ illustrated in FIG. 22 is a regression line (intercept b₃) obtained by performing the feature data extracting operation on the frequency spectrum curve C₃ at Step S59.

The operations performed at Step S60 to Step S62 are respectively identical to the operations performed at Step S10 to Step S12 illustrated in FIG. 4.

As described above, according to the second embodiment of the present invention, with respect to a frequency spectrum that has been obtained by analyzing the frequencies of received ultrasonic sound waves, attenuation correction is performed so that there is a decrease in the contribution of attenuation, which occurs due to the reception depth and the frequency of ultrasonic sound waves. Then, the feature data of a specimen is extracted by performing approximation. Subsequently, based on the extracted feature data of the specimen and based on the feature data of a plurality of known specimens, the tissue characterization of a predetermined area of the specimen is determined. Hence, without having to make use of the strain amount or the degree of elasticity of the body tissues, it becomes possible to make clear distinction between different tissues. As a result, tissue characterizations can be distinguished with accuracy and the measurement result can be enhanced in terms of reliability.

Moreover, according to the second embodiment, since attenuation correction is performed with respect to the frequency spectrum, it becomes possible to eliminate the effect of attenuation that occurs during the propagation of ultrasonic sound waves. That makes it possible to determine tissue characterizations with a higher degree of accuracy.

### Third Embodiment

In a third embodiment of the present invention, the tissue characterization determining operation performed by a tissue characterization determining unit is different than the first embodiment. The configuration of an ultrasonic diagnosis apparatus according to the third embodiment is same as the configuration of the ultrasonic diagnosis apparatus 1 according to the first embodiment. Thus, in the following explanation, the constituent elements identical to those in the ultrasonic diagnosis apparatus 1 are referred to by the same reference numerals.

The tissue characterization determining unit 43 forms new populations by adding the feature data (a, b, c) to each of the groups G_{μ}, G_{ν}, and G_{ρ} that constitute the tissue characterizations μ₀, ν₀, and ρ₀, respectively, (see FIG. 12). Then, for each set of feature data, the tissue characterization determining unit 43 obtains the standard deviation of the data constituting each tissue characterization.

Subsequently, the tissue characterization determining unit 43 calculates the difference between the standard deviation of each set of feature data of the groups G_{μ}, G_{ν}, and G_{ρ} in the original populations formed of only known specimens and the standard deviation of each set of feature data of the groups G_{μ}, G_{ν}, and G_{ρ} in the new population obtained by adding the new specimen to each group (hereinafter, the difference is simply referred to as “standard deviation difference”); and determines that the tissue characterization corresponding to the group including the feature data having the smallest standard deviation difference is the tissue characterization of the specimen.

Herein, alternatively, the tissue characterization determining unit 43 can also calculate the standard deviation difference only with respect to the standard deviations of those sets of feature data which are selected in advance from a plurality of sets of feature data. In this case, the selection of sets of feature data can either be arbitrarily done by the operator or be automatically done in the ultrasonic diagnosis apparatus 1.

Alternatively, the tissue characterization determining unit 43 can also calculate, for each group, values by adding an appropriate weight to the standard deviation differences of all sets of feature data; and then determine that the tissue characterization corresponding to the group having the smallest calculated value is the tissue characterization of the specimen. In this case, for example, if the gradient a, the intercept b, and the intensity c represent feature data; then the tissue characterization determining unit 43 makes use of weights wₐ, w_{b}, and w_{c}, respectively, and calculates “wₐ·(standard deviation difference of a)+w_{b}·(standard deviation difference of b)+w_{c}·(standard deviation difference of c)”. Then, based on the calculated value, the tissue characterization determining unit 43 determines the tissue characterization of the specimen. Herein, the weights wₐ, w_{b}, and w_{c} can either be arbitrarily set by the operator or be automatically set in the ultrasonic diagnosis apparatus 1.

Still alternatively, the tissue characterization determining unit 43 can also calculate, for each group, the root square of the value obtained by adding an appropriate weight to the squares of the standard deviation differences of all sets of feature data; and then determine that the tissue characterization corresponding to the group having the smallest square root is the tissue characterization of the specimen. In this case, for example, if the gradient a, the intercept b, and the intensity c represent feature data; then the tissue characterization determining unit 43 makes use of weights w′ₐ, w′_{b}, and w′_{c}, respectively, and calculates “{w′ₐ·(standard deviation difference of a)²+w′_{b}·(standard deviation difference of b)²+w′_{c}·(standard deviation difference of c)²}^{1/2}”. Then, based on the calculated value, the tissue characterization determining unit 43 determines the tissue characterization of the specimen. Herein too, the weights w′ₐ, w′_{b}, and w′_{c} can either be arbitrarily set by the operator or be automatically set in the ultrasonic diagnosis apparatus 1.

As described above, according to the third embodiment, in an identical manner to the first embodiment, tissue characterizations can be distinguished with accuracy and the measurement result can be enhanced in terms of reliability. Moreover, it becomes possible to eliminate the effect of attenuation that occurs during the propagation of ultrasonic sound waves. That makes it possible to determine tissue characterizations with a higher degree of accuracy.

In the third embodiment, it is explained that the tissue characterization determining unit 43 determines tissue characterizations based on the changes in the standard deviation difference of each set of feature data between the original population and the new population that is obtained by adding a new specimen to the original population. However, that is only an example. Alternatively, for example, the tissue characterization determining unit 43 can determine tissue characterizations based on the changes in the average of each set of feature data between the original population and the new population that is obtained by adding a new specimen to the original population.

### Fourth Embodiment

In a fourth embodiment of the present invention, the tissue characterization determining operation performed by a tissue characterization determining unit is different than the first embodiment. The configuration of an ultrasonic diagnosis apparatus according to the fourth embodiment is same as the configuration of the ultrasonic diagnosis apparatus 1 according to the first embodiment. Thus, in the following explanation, the constituent elements identical to those in the ultrasonic diagnosis apparatus 1 are referred to by the same reference numerals.

The tissue characterization determining unit 43 calculates the probability belonging to each tissue characterization by referring to the distance between the specimen point in the feature data space and the average point of known specimens. More particularly, in the case of the feature data space (b, c) illustrated in FIG. 12, the distances d_{μ}, d_{ν}, and d_{ρ} from the specimen point Sp to the points μ₀, ν₀, and ρ₀, respectively, are used to calculate the probability belonging to each tissue characterization. The setting is so done that smaller the distance, greater is the probability belonging to each known specimen. For example, with λ=100(α⁻¹+β⁻¹+γ⁻¹) (%), it can be defined that the probability belonging to a tissue characterization A is λ/α (%), the probability belonging to a tissue characterization B is λ/β (%), and the probability belonging to a tissue characterization C is λ/γ (%).

In the fourth embodiment, when the display unit 7 displays a determination result display image, the probability belonging to each tissue characterization is displayed in the information displaying portion. For example, when the display unit 7 displays the determination result display image 200, the following determination result is displayed in the information displaying portion 201: “probability of tissue characterization μ=60%, probability of tissue characterization ν=5%, and probability of tissue characterization ρ=35%”.

As described above, according to the fourth embodiment, in an identical manner to the first embodiment, tissue characterizations can be distinguished with accuracy and the measurement result can be enhanced in terms of reliability. Moreover, it becomes possible to eliminate the effect of attenuation that occurs during the propagation of ultrasonic sound waves. That makes it possible to determine tissue characterizations with a higher degree of accuracy.

### Fifth Embodiment

In a fifth embodiment of the present invention, the tissue characterization determining operation performed by a tissue characterization determining unit is different than the first embodiment. The configuration of an ultrasonic diagnosis apparatus according to the fifth embodiment is same as the configuration of the ultrasonic diagnosis apparatus 1 according to the first embodiment. Thus, in the following explanation, the constituent elements identical to those in the ultrasonic diagnosis apparatus 1 are referred to by the same reference numerals.

FIG. 23 is a diagram explaining the overview of the tissue characterization determining operation performed by the tissue characterization determining unit 43 according to the fifth embodiment. In the feature data space illustrated in FIG. 23, the horizontal axis represents the post-attenuation-correction intercept b and the vertical axis represents the post-attenuation-correction intensity c. The feature data space is grouped into areas depending on the tissue characterizations. The tissue characterization determining unit 43 determines the tissue characterization according to the position of the specimen. In FIG. 23, it is illustrated that a point Sp′ lies in a group G_{ν}′ (in which ν is the tissue characterization). In this case, the tissue characterization determining unit 43 determines that ν is the tissue characterization of the area of concern.

As described above, according to the fifth embodiment, in an identical manner to the first embodiment, tissue characterizations can be distinguished with accuracy and the measurement result can be enhanced in terms of reliability. Moreover, it becomes possible to eliminate the effect of attenuation that occurs during the propagation of ultrasonic sound waves. That makes it possible to determine tissue characterizations with a higher degree of accuracy.

Although the invention has been described with respect to the first to fifth embodiments for a complete and clear disclosure, the appended claims are not to be thus limited but are to be construed as embodying all modifications and alternative constructions that may occur to one skilled in the art that fairly fall within the basic teaching herein set forth.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. An ultrasonic diagnosis apparatus that transmits ultrasonic sound waves to a specimen and receives ultrasonic sound waves reflected from the specimen, and that determines tissue characterization of the specimen based on the received ultrasonic sound waves, the ultrasonic diagnosis apparatus comprising:
a frequency analyzing unit that analyzes frequencies of the received ultrasonic sound waves and calculates a frequency spectrum;
a feature data extracting unit that performs attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracts feature data of the specimen;
a storing unit that is used to store feature data of frequency spectrums, each being extracted based on ultrasonic sound waves reflected from one of a plurality of known specimens, and tissue characterizations of the known specimens in a corresponding manner; and
a tissue characterization determining unit that determines tissue characterization of a predetermined area of the specimen by referring to feature data, which is stored by the memory unit in a corresponding manner to the tissue characterizations of the plurality of known specimens, and by referring to the feature data extracted by the feature data extracting unit.

2. The ultrasonic diagnosis apparatus according to claim 1, wherein the feature data extracting unit includes
an approximating unit that performs the approximation with respect to the frequency spectrum calculated by the frequency analyzing unit and extracts pre-correction feature data as feature data prior to performing the attenuation correction; and
an attenuation correcting unit that performs the attenuation correction with respect to the pre-correction feature data extracted by the approximating unit, and extracts feature data of the frequency spectrum.

3. The ultrasonic diagnosis apparatus according to claim 1, wherein the feature data extracting unit includes
an attenuation correcting unit that performs the attenuation correction with respect to the frequency spectrum; and
an approximating unit that performs the approximation with respect to the frequency spectrum corrected by the attenuation correcting unit and extracts feature data of the frequency spectrum.

4. The ultrasonic diagnosis apparatus according to claim 1, wherein, greater the reception depth of ultrasonic sound waves, greater is the extent of correction performed by the attenuation correcting unit.

5. The ultrasonic diagnosis apparatus according to claim 2, wherein the approximating unit performs polynomial approximation with respect to the frequency spectrum by means of regression analysis.

6. The ultrasonic diagnosis apparatus according to claim 5, wherein the approximating unit performs linear approximation with respect to the frequency spectrum and extracts a plurality of sets of feature data that include at least two components from among a gradient of the linear expression, an intercept of the linear expression, and an intensity that is determined using the gradient, the intercept, and a specific frequency included in the frequency band of the frequency spectrum.

7. The ultrasonic diagnosis apparatus according to claim 6, wherein the attenuation correcting unit performs correction with respect to at least the gradient and the intensity.

8. The ultrasonic diagnosis apparatus according to claim 6, wherein
the memory unit stores therein the average of each set of feature data present in groups classified on the basis of tissue characterizations of the plurality of known specimens, and
the tissue characterization determining unit sets a feature data space, which has at least one of the sets of feature data as component, and determines tissue characterization of the specimen based on the distance in the feature data space from a specimen point, for which coordinates in the feature data space indicate sets of feature data serving as components of the feature data space from among the sets of feature data of the frequency spectrum of the specimen, to a known specimen average point, for which coordinates in the feature data space indicate the averages of sets of feature data serving as components of the feature data space from among the sets of feature data in the groups of the known specimens.

9. The ultrasonic diagnosis apparatus according to claim 1, wherein the tissue characterization determining unit calculates standard deviation of feature data in a population, which is obtained by adding the feature data of the specimen in the groups divided on the basis of tissue characterizations of the plurality of known specimens, and sets tissue characterization of the specimen to tissue characterization corresponding to a group that has the smallest difference between the standard deviation and standard deviation of feature data in the groups.

10. The ultrasonic diagnosis apparatus according to claim 1, further comprising:
a signal amplifying unit that amplifies reception signals of ultrasonic sound waves received from the specimen; and
a B-mode-display image data generating unit that generates B-mode-display image data by converting the amplitude of the reception signals that have been amplified by the signal amplifying unit into luminance, wherein
with respect to signals to be output to the B-mode-display image data generating unit, the signal amplifying unit performs amplification by varying gain depending on reception depth, while with respect to signals to be output to the frequency analyzing unit, the signal amplifying unit performs amplification with a constant gain.

11. The ultrasonic diagnosis apparatus according to claim 10, wherein, up to a predetermined reception depth, there is a monotonic increase in the gain with respect to signals to be output to the B-mode-display image data generating unit.

12. The ultrasonic diagnosis apparatus according to claim 1, further comprising:
a B-mode-display image data generating unit that generates B-mode-display image data by converting amplitude of reception signals of ultrasonic sound waves into luminance;
a determination-result-display image data generating unit that generates visual information corresponding to the feature data of the specimen and generates determination-result-display image data, which is used in displaying tissue characterization of the specimen, by referring to the visual information that has been generated, the B-mode-display image data generated by the B-mode-display image data, and a determination result of the tissue characterization determining unit; and
a display unit that displays an image corresponding to the determination-result-display image data generated by the determination-result-display image data generating unit.

13. The ultrasonic diagnosis apparatus according to claim 12, wherein
the determination-result-display image data contains a tissue characterization weighted image in which the tissue characterization determined by the tissue characterization determining unit is highlighted, and
the determination-result-display image data generating unit generates the tissue characterization weighted image by substituting an area determined to be predetermined tissue characterization by the tissue characterization determining unit for a feature data image that has the visual information corresponding to the feature data of the specimen.

14. The ultrasonic diagnosis apparatus according to claim 12, wherein
the determination-result-display image data contains a tissue characterization weighted image in which the tissue characterization determined by the tissue characterization determining unit is highlighted, and
the determination-result-display image data generating unit generates the tissue characterization weighted image by substituting an area other than an area determined by the tissue characterization determining unit to be a predetermined tissue characterization for a feature data image that has the visual information corresponding to the feature data of the specimen.

15. The ultrasonic diagnosis apparatus according to claim 12, wherein
the determination-result-display image data contains a tissue characterization weighted image in which the tissue characterization determined by the tissue characterization determining unit is highlighted, and
the determination-result-display image data generating unit weights mutually-corresponding pixel values in the B-mode display image and in a feature data image that has the visual information corresponding to the feature data of the specimen, and generates the tissue characterization weighted image that has averages of the weighted pixels values as pixel values.

16. The ultrasonic diagnosis apparatus according to claim 12, wherein the visual information indicates variables constituting a color space.

17. An operation method of an ultrasonic diagnosis apparatus that transmits ultrasonic sound waves to a specimen and receives ultrasonic sound waves reflected from the specimen, and that determines tissue characterization of the specimen based on the received ultrasonic sound waves, the operation method comprising:
calculating that includes analyzing frequencies of the received ultrasonic sound waves and calculating a frequency spectrum by a frequency analyzing unit;
extracting that includes performing attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracting feature data of the specimen by a feature data extracting unit; and
determining that includes determining tissue characterization of a predetermined area of the specimen by referring to feature data of frequency spectrums, each being extracted based on ultrasonic sound waves reflected from one of a plurality of known specimens, and by referring to the feature data extracted by the feature data extracting unit by a tissue characterization determining unit.

18. A non-transitory computer readable recording medium with an executable program stored thereon, wherein the program instructs a processor to perform:
calculating that includes analyzing frequencies of the received ultrasonic sound waves and calculating a frequency spectrum by a frequency analyzing unit;
extracting that includes performing attenuation correction and approximation with respect to the frequency spectrum calculated by the frequency analyzing unit so that there is a decrease in the contribution of attenuation, which occurs due to the reception depth and the frequency of ultrasonic sound waves being propagated, and extracting feature data of the specimen by a feature data extracting unit; and
determining that includes determining tissue characterization of a predetermined area of the specimen by referring to feature data of frequency spectrums, each being extracted based on ultrasonic sound waves reflected from one of a plurality of known specimens, and by referring to the feature data extracted by the feature data extracting unit by a tissue characterization determining unit.
